(19) United States
(12) Patent Application Publication
(10) Pub. No.: US 2007/0238907 A1
(43) Pub. Date: Oct. 11, 2007

(54) Method for removing sulfur compounds from an alcohol stream

(76) Inventors: Thomas P. Binder, Decatur, IL (US); Joseph R. Beggin, Warrensburg, IL (US); Ahmad K. Hilaly, Springfield, IL (US); Lawrence P. Karcher, Decatur, IL (US); Leif P. Solheim, Decatur, IL (US); John G. Soper, Mt. Zion, IL (US); Brad Lee Zenthoefer, Decatur, IL (US)

Correspondence Address: Craig G. Cochenour, Esq.;Buchanan Ingersoll & Rooney PC, One Oxford Centre, 301 Grant Street, 20th Floor, Pittsburgh, PA, 15219, US

(21) Appl. No.: 11/784,052

(22) Filed: Apr. 5, 2007

Related U.S. Application Data
(60) Provisional application No. 60/789,470, filed on Apr. 5, 2006.
(60) Provisional application No. 60/855,017, filed on Oct. 27, 2006.

(51) Int. Cl. C07C 29/74; C12P 7/06

(52) U.S. Cl. 568/913; 435/161

(57) ABSTRACT

Processes, apparatus, and systems for purifying alcohol streams by reducing the concentration of sulfur compounds in those alcohol streams are presented herein. The invention is exemplified by reduction of sulfur dioxide, sulfate ion, and sulfite ion in an ethanol stream, but is applicable for the removal of other sulfur compounds from other alcohol streams.

### CROSS-REFERENCE TO RELATED APPLICATIONS

This Application claims the benefit of priority to pending U.S. Provisional Patent Application Ser. No. 60/789,470, filed on Apr. 5, 2006, and to pending U.S. Provisional Patent Application Ser. No. 60/855,017, filed on Oct. 27, 2006, entitled “Method for Removing Sulfur Compounds from an Alcohol Stream” and having the same named inventors. U.S. Provisional Patent Application Ser. Nos. 60/789,470 and 60/855,017 are incorporated by reference into this Application as if fully written herein.

### BACKGROUND OF THE INVENTION

### 1. Field of the Invention

Ethanol is widely used in industry as a solvent in the synthesis of paints, pharmaceuticals and intermediaries, cosmetics, perfumes, and other products. Anhydrous ethanol (that is, dewatered ethanol) is also an important component in alternative fuels. Alternative fuels may be created through combination of ethanol with, for example gasoline and other fossil fuel distillate components. These alternative fuels may include, for example, E10 gasohol or E85 gasohol (having 10% and 85% anhydrous ethanol, respectively), though of course the percentage of ethanol may vary to suit a desired application. Anhydrous ethanol can also be used as an important oxygenic additive in lead-free gasoline.

Because of the complexity of modern applications of ethanol, methanol, and other alcohol streams, it is desirable to provide such streams in as high a purity as possible. One common impurity in alcohol streams is sulfur. This sulfur may be present, for instance, as sulfate anions and compounds, sulfite anions and compounds, or sulfur dioxide. Those skilled in the art will recognize that other sulfur compounds may be present in alcohol streams.

Sulfur compounds may be present in ethanol streams for a variety of reasons. For example, they may be present due to their initial presence in the raw materials used to create ethanol streams and/or due to introduction of sulfur compounds during processing to obtain ethanol. Ethanol streams produced from corn by a wet-milling process may include, for example, at least about 8 ppm (that is, about 8 mg/liter) of sulfur as sulfur dioxide. Ethanol streams produced from corn by a dry-milling process may include, for example, at least about 2 ppm of sulfur as sulfur dioxide.

It would be desirable to provide a method, apparatus, and system for reduction of sulfur compounds in ethanol, methanol, and other alcohol streams.

### BRIEF SUMMARY OF THE INVENTION

Described herein are novel processes, apparatus, and systems for purifying alcohol streams by reducing the concentration of sulfur compounds in those alcohol streams. The invention is exemplified by reduction of sulfur dioxide, sulfate ion, and/or sulfite ion in an ethanol stream, but is applicable for the removal of other sulfur compounds from other alcohol streams. In one embodiment, short-chain alcohol streams are purified.

An embodiment includes a method of removing at least one sulfur compound from an alcohol. This method may include contacting an amount of alcohol including at least one sulfur compound with at least one material selected from an anion ion exchange resin, an aluminum silicate clay, alumina silicate (alumina), activated carbon, smectite clay, barium salt and mixtures of those things, waiting for a time sufficient to allow the material to reduce the amount of sulfur compound to a predetermined amount, and recovering alcohol including at least one sulfur compound in an amount no greater than the predetermined amount.

In another embodiment, the invention includes a system for producing reduced sulfur ethanol. Such a system may include a grain processing facility configured to add a sulfur containing compound to a grain feed stream, and/or to form a grain feed stream inherently containing at least one sulfur containing compound; a grain fermenting facility configured to ferment ethanol from a sulfur containing feed stream to form a fermentation broth; an enrichment facility configured to obtain an enriched ethanol fraction from a fermentation broth, wherein the enriched ethanol fraction contains at least 4 ppm of sulfur containing compounds; and a sulfur removal facility configured to remove at least a portion of sulfur containing compound from the enriched ethanol fraction, where the sulfur removal facility is configured with an apparatus to remove at least a portion of sulfur containing compound from the enriched ethanol. The removal may be accomplished by other methods disclosed in this application.

In a still further embodiment, a sulfur compound reducing material is provided in a slurry, continuous flow bed, countercurrent extractor, moving bed, stationery bed, automated ion exchange system, an ion exchange column, impregnated filter, or combination thereof.

In a further embodiment, the amount of sulfur compounds in an alcohol stream may be reduced to below 4 ppm, 3 ppm, 2 ppm, 1 ppm, 0.5 ppm, or 0.1 ppm. In a still further embodiment of the invention, the alcohol stream includes more than 4 ppm of sulfur compounds prior to treatment. In another embodiment of the invention, sulfur compounds for removal are selected from sulfur dioxide, sulfate anion, sulfite anion, and mixtures thereof.

In a further embodiment, a material used for sulfur compound removal is an aluminum silicate clay. Aluminum silicate clay may be, for example, but is not limited to, a montmorillonite clay, a bentonite clay, a zeolite clay, or a zeolite-like clay. A bentonite clay may be a calcium bentonite clay.

In a further embodiment, a material used for sulfur compound removal is an ion exchange resin. In one embodiment, an ion exchange resin is macro porous and is a weak base anion exchanger, a strong base type 1 anion exchanger, or a strong base type 2 anion exchanger. An ion exchange resin may be, for example, but is not limited to, Mitsubishi WA30, Mitsubishi DCA11, Lewatit S4228, Lewatit S4528, Amberlyst A26, Amberlyst A21, Lewatit Mono+MP500, Dowex 22, Dowex 66, Mitsubishi PA412, and Mitsubishi PA312.

In a further embodiment, a material used for sulfur compound removal is a barium salt. A barium salt may be, for example, but is not limited to, barium hydroxide, barium carbonate, or mixtures of the two. Barium salts for use in the invention may have greater solubility in alcohol (for example, in ethanol) than barium sulfate has in ethanol.

Alcohols for inclusion in purification processes of the invention may include, for example, ethanol, methanol, or mixtures thereof. In a preferred embodiment, the alcohol is ethanol.

A further embodiment includes an ethanol comprising less than about 4 ppm sulfur compounds.

### BRIEF DESCRIPTION OF THE DRAWINGS

FIG. 1 shows a typical corn dry grind ethanol process including sulfur removal, as encompassed in an embodiment of the invention.

FIG. 2 shows a typical flow diagram of a wet milling process for production of starch. Areas of potential introduction of sulfur compounds are shown.

FIG. 3 shows a flow diagram of a typical ethanol production process using starch from a wet mill. Proposed areas of possible sulfur removal are shown.

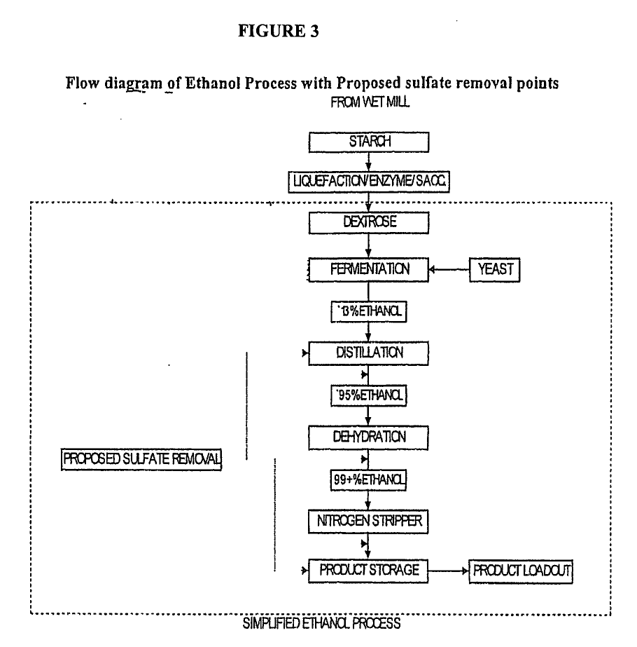

### DETAILED DESCRIPTION OF THE INVENTION

Unless otherwise indicated, the terms in this application shall have their art-accepted meanings. In an effort to aid understanding of the invention, a number of terms are defined below.

As used herein, the term “smectite clay” means a clay having a three-layer crystalline structure of one alumina and two silica layers. Smectite clays are characterized by hydrational swelling and colloidal characteristics.

As used herein, the term “bentonite clay” means a colloidal clay composed primarily of montmorillonte but also including other smectite clays. Both sodium bentonite and calcium bentonite exist. Sodium bentonite has a high swelling capacity in water, and calcium bentonite does not.

As used herein, the term “zeolite” means a hydrated silicate of aluminum and either sodium or calcium or both, including framework silicates with interlocking tetrahedrons of SiO₄ and AlO₄. Zeolites for use in the invention may be natural or artificial. Zeolites may be natrolites, heulandites, or Chabazites. “Zeolite-like materials” include minerals and compounds with structures and/or properties similar to those of zeolites. Zeolite-like materials include phosphates and silicates. Representative natural phosphates include kehoeite, pahasapaite and tiptopite. Representative natural silicates include hsianghualite, lovdarite, viseite, partheite, prehnite, roggianite, apophyllite, gyrolite, maricopaite, okenite, tacharanite and tobermorite.

Zeolites are typically framework silicates including interlocking tetrahedrons of SiO₄ and AlO₄. Generally the ratio (Si+Al)/O equals ½. The alumino-silicate structure is negatively charged and attracts the positive cations that reside within. Unlike most other tectosilicates, zeolites have large vacant spaces or cages in their structures that allow space for large cations. These large cations may include, for example, but are not limited to, sodium, potassium, barium, and calcium, as well as relatively large molecules and cation groups including water, ammonia, carbonate ions, and nitrate ions. In some zeolites the spaces are interconnected and form long, wide channels of varying sizes (where size depends on the mineral structure). These channels allow the easy movement of the resident ions and molecules into and out of the structure. Zeolites are typically characterized by their ability to lose and absorb water without damage to their crystal structures. The large channels are one explanation for the consistent low specific gravity of zeolites.

As used herein, the terms “montmorillonite clay” and “montmorillonite” mean a type of clay having an approximate composition:

[(½Ca,Na)_{0.7}(Al,Mg,Fe)₄(Si,Al)₈O₂₀(OH)₄₇nH₂0].

As stated in Hawley's Condensed Chemical Dictionary (11th Ed., 1987) (Sax and Lewis, eds.), incorporated by reference herein, montmorillonite is a major component of bentonite.

As used herein, the term “short chain alcohol” means an alcohol having one to six carbons in its longest carbon chain.

The present teachings encompass providing an alcohol stream that includes one or more sulfur compounds. Sulfur compounds include, for example, but are not limited to, elemental sulfur, sulfur dioxide, hydrogen sulfide, sulfur trioxide, and compounds and ionic species containing sulfate, sulfite, and/or sulfide. Alcohol streams are preferably short-chain alcohol streams. Ethanol streams and methanol streams are particularly preferred.

Sulfur removal from an alcohol as taught herein may be performed before or after dewatering of that alcohol. Sulfur removal after dewatering is preferred.

Provided sulfur-bearing alcohol streams may be treated to reduce the amount of sulfur that they include by the methods described herein. In one aspect, a sulfur-bearing alcohol stream is contacted with an ion exchange resin over a period of time. An ion exchange resin may be, for example, but is not limited to, macro porous and a weak base anion exchanger, a strong base type 1 anion exchanger, or a strong base type 2 anion exchanger. Gel type resins are less preferred. Exemplary resins for use in the invention include, for example, but are not limited to, Mitsubishi WA30, Mitsubishi DCA11, Lewatit S4228, Lewatit S4528, Amberlyst A26 (Rohm and Haas), Amberlyst A21 (Rohm and Haas), Lewatit Mono+MP500, Dowex 22 (Dow Chemical Company), Dowex 66 (Dow Chemical Company), Mitsubishi PA412, and Mitsubishi PA312.

Those skilled in the art will recognize that gel resins typically include lower cross-linked dense beads, which have high capacity and high breaking weights. Others recognize gel-type resins that have no permanent pore structures. Their pores are generally considered to be quite small, usually not greater than 30 Angstroms, and are referred to as gelular pores or molecular pores. The pore structures are determined by the distance between the polymer chains and crosslinks, which vary with the crosslink level of the polymer, the polarity of the solvent, and the operating conditions used with the resin. Gel type resins are typically translucent.

Those skilled in the art will further recognize that macroporous resins are typically lower in capacity than gel resins, but have a higher resistance to fouling and are more resistant to osmotic shock attrition. Macroporous resins are made of two continuous phases, a continuous pore phase and a continuous gel polymeric phase. The polymeric phase is typically structurally composed of small spherical microgel particles agglomerated together to form clusters. The clusters are, in turn, fastened together at the interfaces, forming interconnecting pores. The increased surface area arises from the exposed surface of the microgel, glued together into clusters. Macroreticular ion exchange resins can be made with different surface areas. These surface areas may range, for example, between 7 to about 1500 m²/g, with average pore diameters ranging from about 50 to about 1,000,000 Angstroms.

Once exhausted, resin used in the manner described herein may be regenerated. For instance, regeneration may be accomplished by a sodium hydroxide and alcohol wash. Although the resin may be included in any of a variety of constructs as described herein, operation in a plurality of ion exchange columns is preferred.

Purification by ion exchange resin may be conducted, for example, at “room temperature” (i.e. about 21-23° C.), though those skilled in the art will appreciate that ion exchange may be conducted at a wide range of temperature and pressure. Those of skill in the art will also appreciate the fact that ion exchange can be implemented at various stages within the ethanol process. Such stages would include: immediately following distillation (˜90-95% ethanol), immediately following dehydration (99+% ethanol), or after nitrogen stripping, or other supplemental purification step. Possible pH values for ion exchange operations as taught herein range from about 1 to about 10. Preferred pH range for ion exchange operations as described herein is between about 8 and 9, though pH values less than 8 are effective.

In a further aspect, sulfur removal is accomplished by mixture of a sulfur-containing alcohol stream with aluminum oxide (alumina), silica, aluminum silica oxide, smectite clay, montmorillonite, bentonite, a zeolite, a zeolite-like material, activated carbon, or mixtures thereof. In one aspect, an alcohol stream containing sulfur compounds is mixed with one of the foregoing materials for a period of time in a slurry, then filtered.

Although applicants do not wish to be bound by theory, it is believed that sulfur compounds in the alcohol stream are either adsorbed to the material or trapped by ion exchange. After a time sufficient to reduce the amount of sulfur compounds to a desired level, the mixture is filtered and more pure alcohol filtrate is removed.

Those skilled in the art will recognize, with the benefit of this disclosure, that temperature is not likely to be critical to this reaction so long as the temperature is not extreme, but that a temperature higher than room temperature is preferred.

The amount of resin suitable to remove a desired amount of sulfate (or other sulfur compound) from an alcohol stream may be determined. For example, the equivalents/liter of sulfate in a given ethanol stream may be determined based on parts per liter of sulfate in the stream. The amount of alcohol treated by a given volume of resin may be determined by the formula:

Volume_{Resin}*Operating Exchange Capacity_{Resin}/(Equivalents of Sulfur Anion/Volume Alcohol)

The total ion exchange capacity of a resin is usually determined and advertised by the manufacturer. The operating capacity is the quantity of ions that a resin will bind at which the product of the resin treatment is acceptable. The operating capacity is usually determined experimentally by the user for the intended application. Those skilled in the art can determine the operating capacity and recognize that system design and operational conditions affect the operating capacity. The total ion exchange capacity often does not match the operating capacity, however the total capacity can be used to estimate amount of material that a resin can process.

For example, an ethanol stream with 11.8 ppm of sulfate has 0.00025 eq/L of sulfate. (This calculation assumes that sulfate is the only sulfur anion present. If additional sulfur is present, the eq/L will be greater.) The weak base anion exchange resin Lewatit S4228 has a stated capacity of 1.8 to 1.9 eq/L, which means that 1 liter of resin could treat up to 7600 L of ethanol. The strong base anion exchange type 2 resin Dowex 22 has a stated capacity of 1.2 eq/L, for a treatment amount of 4800 L ethanol. The strong base anion exchange type 1 resin PA316, from Itochu, has a stated capacity of 1.3 eq/L, resulting in a potential treatment amount of 5200 L of ethanol. More accurate values can be calculated if the operating capacity of each resin is known.

Ion exchange resin procedures usually include at least two modes of operation, the loading (service) cycle and the regeneration cycle. The service cycle, as it pertains to the present invention, relates to the time which the column is processing feed ethanol and removing the sulfur compounds from it. This aspect will be sufficiently covered elsewhere in this document.

After the service cycle the resin is exhausted and should be regenerated for re-use. Regeneration may be performed, for example, by aqueous sodium hydroxide, sodium carbonate, potassium hydroxide, or other compounds.

When using resins in alcohol or oil matrices, however, it is preferred that one does not introduce water to the system. Regeneration in these cases may be conducted using varying concentrations of sodium hydroxide, ammonium hydroxide, and other compounds in ethanol/water mixtures having ratios of ethanol to water of, for example, 0:100, 50:50, 90:10, 99+:1. Preferred regenerative compositions may have, for example, a 5% (by volume) sodium hydroxide solution in an ethanol/water mixture having an ethanol to water ratio of 0.5 to 99.5.

The employment of an aqueous regeneration scheme as taught herein may include four steps, though those skilled in the art of ion exchange will recognize that steps may be added, modified, or removed: 1) the evacuation of product ethanol (with water), known, in the corn sweetener industry, as the “sweeten off” step, 2) the actual regeneration step, 3) the regeneration rinse step, and 4) the evacuation of rinse water (with feed ethanol), known as the “sweeten on” step.

The sweeten off step may use, for example, between about 1 to 3 bed volumes (BV) of water to evacuate product ethanol, though more or less water may be used if desired. In one embodiment, about 2 BV of water are used to get the column effluent from about 99% ethanol to less than about 5% ethanol. Circulation rate of the water may also vary, with longer circulation rates generally leading to removal of more column effluent. In one embodiment, the circulation rate is between about 1 to about 5 BV/hour, with about 3 to about 4 BV/hour being preferred. Those skilled in the art will recognize, for instance, that lower water percentage in the sweeten off step leads to lower efficiency of regeneration. For example, a solution that is about 90% water may lead to about 70% efficiency.

The amount of aqueous regeneration material to be used in the regeneration step may also vary. For example, between about 2 BV to about 7 BV may be used, with 5 BV preferred. In one embodiment the aqueous regeneration material is a 5% sodium hydroxide solution. Those skilled in the art will recognize that the flow rate may be varied. A flow rate of between about 3 to about 6 BV/hour is preferred, with about 5 BV/hour being particularly preferred. Other bases, either in aqueous or organic solvents could also be used.

The regeneration rinse step is preferably conducted with sufficient flow to remove the regeneration reagent from the bed; this flow varies depending on reagent. The sweeten on step may use, for example, between about 1 BV and about 6 BV of feed ethanol, where the feed ethanol has an ethanol to water ratio of between about 90:10 to about 99.5:0.5. Flow rate may vary between about 3 BV/hour to about 6 BV/hour. Preferred amounts include 2.7 BV of feed ethanol (99+%) to get the column effluent from 0% ethanol to 99+% ethanol, at 3.6 BV/HR. The resin is then placed back in service and is used again. Those of ordinary skill can appreciate the fact that these conditions are not meant, in any way, to limit the scope of embodiments herein. Other conditions may be used by those skilled in the art.

In a further aspect of the invention, removal of sulfur compounds from an alcohol stream is accomplished by precipitation of sulfur compounds as barium sulfate. This may be accomplished by treatment of a sulfur-containing alcohol stream with a barium compound. Suitable barium compounds include, for example, but are not limited to, barium hydroxide and barium carbonate. Precipitation may be accomplished with compounds including other Group II elements that result in formation of sulfur compounds with little or no solubility in alcohol, particularly ethanol. Suitable compounds including Group II elements may include strontium or radium. For example, hydroxides and carbonates of radium or strontium may be useful in the invention.

Sulfur compounds may be removed from an alcohol stream, for example, by mixing the alcohol stream with barium hydroxide in a slurry for a period of time. Because barium sulfate is either insoluble or very sparingly soluble in alcohol barium sulfate will precipitate from the mixture. The mixture may be filtered, and the purified alcohol filtrate may be collected. In one aspect, mixture and filtrate are accomplished simultaneously by use of a continuous filter, or by use of a filter impregnated with a barium compound.

In a further aspect of the invention, removal of sulfur compounds from an alcohol stream is accomplished by contacting an alcohol stream that contains one or more sulfur compounds with one or more metals. A metal surface can remove both sulfate and other sulfur compounds that can be oxidized to sulfate. Metals that may be used include, but are not limited to, iron, copper, or zinc. The contact between the metal and the alcohol stream can be accomplished by the addition of pure metals, metal alloys, or combinations thereof to an alcohol stream. The metal is then separated from the alcohol by filtration, evaporation, or another method known to those skilled in the art. In a further embodiment, an alcohol stream is passed through a bed of metal particles or metal wool.

Like other embodiments of the invention, this embodiment may be used, for example, to meet a maximum sulfate specification in fuel alcohol. It may also be used to meet a specification limiting sulfur compounds that may be converted to sulfate by oxidation; this may occur, for example, during a peroxide conversion sulfate test.

Removal of sulfur compounds from an alcohol stream using metal contact may be used in conjunction (either simultaneously or successively) with other methods described herein. For example, metal contact may be used in conjunction with an ion exchange resin used to reduce sulfates. In the event that metal ions leach during this process, they may be removed using any method known to those of skill in the art. For example, leached metal may be removed with a cation exchange resin or a chelating resin.

In a further embodiment of the invention, metals used to remove sulfur compounds are attached to substrates, including but not limited to non-metallic substrates or ion exchange resins. Those skilled in the art will recognize, with the teachings herein, that this method may be used with a variety of metals and on a variety of alcohol streams.

With the benefit of this disclosure, the period of time necessary to achieve desired reduction of sulfur in the methods taught herein may be readily determined. Generally, longer treatment times lead to greater removal of sulfur compounds, though a point of diminishing return for time invested will eventually be reached.

Although methods taught herein may be useful in treatment of alcohol streams bearing any initial sulfur load, in a preferred embodiment of the invention, the alcohol stream to be treated includes at least 1 ppm sulfur compounds, at least 2 ppm sulfur compounds, 3 ppm sulfur compounds, at least 4 ppm sulfur compounds, at least 5 ppm sulfur compounds, at least 6 ppm sulfur compounds, at least 7 ppm sulfur compounds, at least 8 ppm sulfur compounds, at least 9 ppm sulfur compounds, at least 10 ppm sulfur compounds, at least 11 ppm sulfur compounds, and at least 12 ppm sulfur compounds.

Methods taught herein may reduce the amount of sulfur compounds in an alcohol stream to at or below a desired level. In various embodiments of the invention, for example, the amount of sulfur compounds is reduced to no more than 4 ppm, no more than 3 ppm, no more than 2 ppm, no more than 1 ppm, and no more than 0.5 ppm.

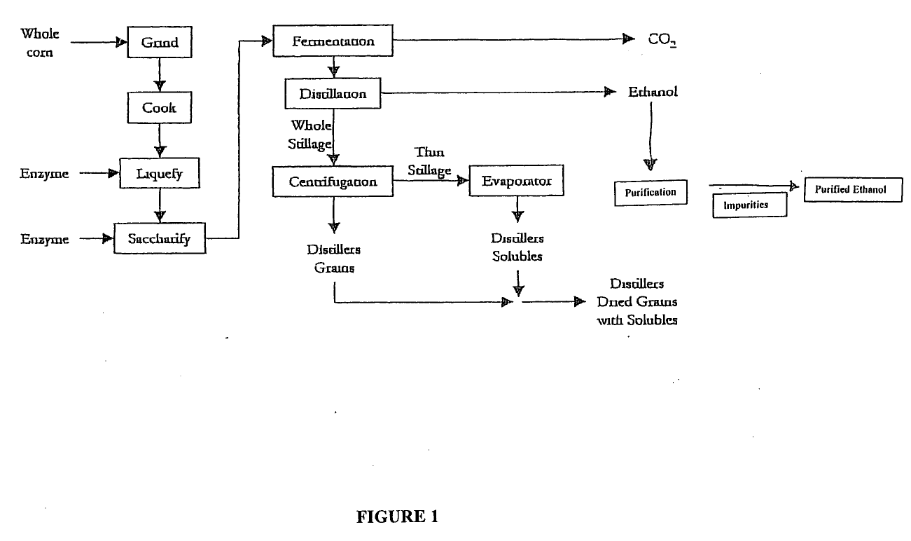

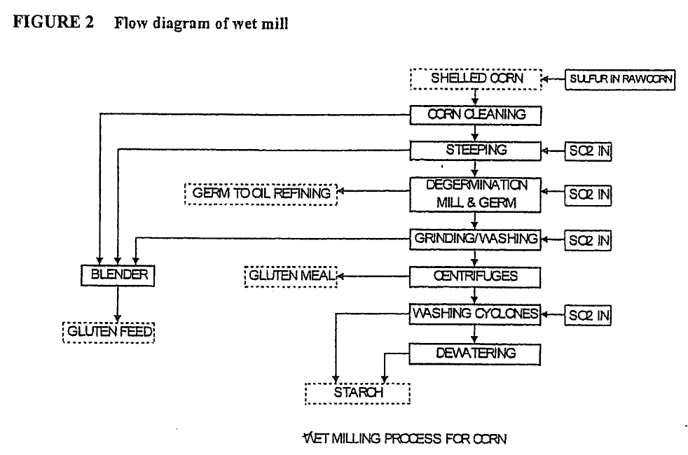

Sulfur compounds may be included in an alcohol stream for a variety of reasons, and the specific mechanism by which a sulfur compound has been introduced to an alcohol stream may not be relevant to determination of the way in which it is removed. Sulfur compounds may be introduced to an ethanol stream, for example, during production of an ethanol stream from corn products in a wet milling plant or in a dry milling plant. Milling processes that may introduce sulfur into an ethanol stream are shown in FIG. 1 and FIG. 2.

Those skilled in the art will recognize that a number of methods exist for measuring the concentration of sulfur in an alcohol stream. For example, one may measure the concentration of sulfur using an ion chromatography column with a conductivity detector. The mobile phase in the column typically is a solution of water, methanol, and sodium hydroxide. Other methods of measuring sulfur compounds in an alcohol stream include ASTM methods D2622-03 (“Wavelength Dispersible X-Ray Fluorescence Spectrometer”) and D5453-03a (“Sulfur Analyzer”).

The methods taught herein may be used alone or in combination. When used in combination, removal methods may be simultaneous (either taking place in a single reaction vessel or in parallel) or serial. Removal steps may be repeated or varied as desired to increase efficacy.

Those skilled in the art will, with the benefit of this disclosure, recognize that there are a variety of ways in which an alcohol stream may be put into contact with the sulfur-removing compositions described herein. For example, a stream may be admixed with a sulfur-removing composition in a slurry, mixing tank, ion exchange column, moving-bed ion exchange device, counter-current ion exchange device, continuous filter, or filter impregnated with the composition. Throughput may be continuous or in a batch process. Where necessary, spent sulfur-removal material may be removed, for example, by filtration, centrifugation or gravity-assisted sedimentation.

### EXAMPLES

The following examples demonstrate aspects of the invention in greater detail. The examples are not intended to limit the scope of the various aspects of the invention.

### Example 1—Removal of Sulfur from Ethanol Using Anion Exchange Resin

Several tests were completed in which a 0.1 L sample of ethanol including about 12 ppm sulfate was placed in a beaker with 0.005 L of anion exchange resin and stirred at room temperature. After about one hour each ethanol sample was tested for sulfate level. A sulfate level of less than 1 ppm (measured by ion chromatography) was achieved in tests with macro porous resins, including in tests with weak base anion resins (for example, Dowex 66, available from the Dow Chemical Company) and in tests with strong base anion resins (for example, Amberlyst A26, available from Rohm and Haas Company, and Dowex 22, available from the Dow Chemical Company). A test with Amberlyst A24, a gel-type resin, did not reduce the sulfate level below 1 ppm.

### Example 2—Removal of Sulfur from Ethanol using Bentonite Clay and Other Adsorbents

Several tests were completed in which a 100 ml sample of 200 proof ethanol containing about 11.7 ppm sulfate (and about 0 ppm sulfite) was combined with 5.0 g of an adsorbent in a 250 ml Pyrex screw cap bottle. The solution was placed in a heated water bath and allowed to run overnight (at least 8 hours) at about 50° C. with stirring. The solution was removed from the bath and run through 1 micron filter paper; the resulting ethyl alcohol filtrate was submitted for ion chromatography analysis of sulfite and sulfate content. Adsorbents used and resulting amounts of sulfite and sulfate are shown in Table 1.

TABLE 1

| | Sulfite | |
|---|---|---|
| Adsorbent | Content (ppm) | Sulfate Content (ppm) |
| Spherical Makall (Silica Gel | Not Detected | 4.6 |
| 0.5–1.5 mm diameter) | | |
| Activated Carbon (Pittsburgh) | Not Detected | 1.2 |
| U.S. Silica F-55 | Not Detected | 10.5 |
| Activated 28 × 50 mesh | Not Detected | 0.2 |
| Alumina Oxide | | |
| Oil Dri Agsorb Ultra Clear | 0.2 | 0.6 |
| 16/30 (Bentonite Clay) | | |

### Example 3—Removal of Sulfur from Ethanol Using Barium Salts

About 0.085 g of barium hydroxide was added to 0.250 L of ethanol and stirred for about one hour. The mixture was filtered using 0.2 micron filter paper. The filtrate was analyzed with ion chromatography. The filtrate contained about 1.9 mg/l of sulfate.

### Example 4—Regeneration of Ion Exchange Column

Regeneration of an ion exchange unit used for sulfur removal from and ethanol stream was performed. About 2.1 bed volumes (BV) of water were circulated at 3.6 BV/hour, reducing the column effluent from 99+% ethanol to less than 0.5% ethanol. About 5 BV of an aqueous solution of 5% sodium hydroxide was circulated at about 5 BV/hour for regeneration. The regeneration rinse step was conducted using 5 BV of Deionized water at a rate of about 5 BV/hour. The sweeten on step was conducted with 2.7 BV of feed ethanol (99+% ethanol) to drive the column effluent from 0% ethanol to 99+% ethanol, at 3.6 BV/HR. The resin was then placed back in service and is used again.

### Example 5—Purification by Electrodialysis and Electrodeionization

In electrodialysis and electrodeionization method, an electrical driving force (voltage) is used to transport ions across ion exchange membranes. Ethanol solutions containing>10 ppm sulfate ions are circulated through an electrodialysis stack. The stack consists of a series of alternating cells made of cation exchange and anion exchange membranes in a parallel array to form compartments. A suitable DC voltage (30-40 volts) is applied across the stack. Sulfate ions permeate through the anion exchange membrane toward the anode resulting in a retentate portion that is essentially free (<0.5 ppm) of sulfate ion. The space between anion membrane and cation membrane are filled up with ion exchange resins or porous ion exchange sheet to facilitate the transport of the sulfate ions at a very low concentration.

### Example 6—Purification by Metal Contact

An experiment was completed in which samples of ethanol were contacted with one of the materials listed in Table 2. The samples were shaken for about one hour and allowed to settle. A portion of liquid from each sample was decanted for analysis. The materials tested were iron powder, copper powder, steel wool, and bronze wool. The dosage was two grams of metal per 70 milliliters of ethanol. The analysis consisted of testing for sulfate and sulfite by ion chromatography before and after oxidation with hydrogen peroxide. Oxidation with hydrogen peroxide was done to convert all sulfur compounds into sulfate.

TABLE 2

| | | As is analysis | | | | Oxidized analysis | |
|---|---|---|---|---|---|---|---|
| | | Acetate | Formate | Sulfite | Sulfate | Sulfite | Sulfate |
| ID | Description | Content | Content | Content | Content | Content | Content |
| 4806-005-1 | Feed | 4.6 | 0.4 | 2.1 | 1.0 | 0.7 | 8.4 |
| 4806-005-A | Elemental Iron Powder | 8.9 | 0.4 | 2.5 | 0.7 | 0.6 | 6.4 |
| 4806-005-B | Elemental Copper Powder | 10.4 | 0.5 | 0.2 | 0.5 | 0.4 | 2.3 |
| 4806-005-C | Steel Wool gr 0000 from Rhodes America | 9.5 | 0.7 | 1.1 | 0.3 | 0.5 | 4.3 |
| 4806-005-D | Bronze Wool from Homax | 11.1 | 0.5 | ND | 0.3 | 0.2 | 1.0 |
| 4806-005-E | Control | 9.2 | 0.4 | 2.2 | 1.0 | 0.5 | 8.1 |
| | | mg/L | mg/L | mg/L | mg/L | mg/L | mg/L |

These results demonstrate a reduction in non-oxidized sulfate concentration from 1 mg/L to 0.3 mg/L and a reduction in oxidized sulfate from 8.4 mg/L to 1.0 mg/L.

Other metals in different combinations may be tested. Loading metal particles or metal wool into a column and passing alcohol through it will demonstrate additional sulfate reduction. The quantity of a metal that is required to reduce the sulfur containing compound level sufficiently would be determined experimentally. Physical and chemical treatments intended to regenerate a saturated adsorbent may also be used, as will physical and chemical treatment of metal surfaces to increase catalytic or absorption properties. These conditions include, but are not limited to, cleaning, abrading, reforming, thermal treatment, oxidation or reduction, acid or base treatment, or other methods. Various metals attached to non-metallic substrates or metal ions bound to ion exchange resins may also be used.

Whereas particular embodiments of the instant invention have been described for purposes of illustration, it will be evident to those persons skilled in the art that numerous variations may be made without departing from the instant invention as defined in the appended claims.

We claim:

1. A method of removing at least one sulfur compound from an alcohol, comprising:
a. contacting a first amount of alcohol comprising at least one sulfur compound with at least one material selected from the group consisting of an anion ion exchange resin, an aluminum silicate clay, alumina silicate, alumina, activated carbon, smectite clay, zinc, copper, brass, iron, a metal, a metal bound to a substrate, a metal bound to an ion exchange resin, barium salt and mixtures thereof;
b. waiting for a time sufficient to allow said material to reduce the amount of said sulfur compound to a predetermined amount of said sulfur compound; and
c. recovering a second amount of alcohol comprising said at least one sulfur compound in an amount no greater than said predetermined amount.

2. A system for producing reduced sulfur ethanol, comprising:
a. a grain processing facility configured to add a sulfur containing compound to a grain feed stream, and/or to form a grain feed stream inherently containing at least one sulfur containing compound;
b. a grain fermenting facility configured to ferment ethanol from said sulfur containing feed stream to form a fermentation broth;
c. an enrichment facility configured to obtain an enriched ethanol fraction from said fermentation broth, wherein said enriched ethanol fraction contains at least 4 ppm of sulfur containing compounds; and
d. a sulfur removal facility configured to remove at least a portion of sulfur containing compound from said enriched ethanol fraction, wherein said sulfur removal facility is configured with an apparatus to remove said portion of sulfur containing compound from said enriched ethanol fraction by the method of claim 1.

3. The method of claim 1 or system of claim 2, wherein said predetermined amount of said sulfur compound is selected from the group consisting of 4 ppm, 3 ppm, 2 ppm, 1 ppm, 0.5 ppm, and 0.1 ppm.

4. The method of claim 1 or system of claim 2, wherein said first amount of ethanol comprises more than 4 ppm of said sulfur compound.

5. The method of claim 1 or 2, wherein said sulfur compound is selected from the group consisting of sulfur dioxide, sulfate anion, and sulfite anion.

6. The method of claim 1 or system of claim 2, wherein said aluminum silicate clay comprises a clay selected from the group consisting of a montmorillonite clay, a zeolite clay, a zeolite-like clay, and combinations thereof.

7. The method of claim 1 or system of claim 2, wherein said aluminum silicate clay comprises a bentonite clay.

8. The method of claim 1 or system of claim 2, wherein said aluminum silicate clay comprises a calcium bentonite clay.

9. The method of claim 1 or system of claim 2, wherein said ion exchange medium is a macro porous resin that comprises at least one of a weak base anion exchanger, a strong base type 1 anion exchanger, or a strong base type 2 anion exchanger.

10. The method of claim 1 or system of claim 2, wherein said ion exchange resin is selected from the group consisting of Mitsubishi WA30, Mitsubishi DCA11, Lewatit S4228, Lewatit S4528, Amberlyst A26, Amberlyst A21, Lewatit Mono+MP500, Dowex 22, Dowex 66, Mitsubishi PA412, and Mitsubishi PA312.

11. The method of claim 1 or system of claim 2, wherein said barium salt is selected from at least one member of the group consisting of barium hydroxide and barium carbonate.

12. The method of claim 1 or system of claim 2, wherein said barium salt has greater solubility in ethanol than barium sulfate has in ethanol.

13. An ethanol comprising less than about 4 ppm sulfur compounds.

14. The method of claim 1, wherein said alcohol is selected from the group consisting of ethanol, methanol, and mixtures thereof.

15. The method of claim 1 or system of claim 2, wherein said at least one material is provided in at least one member of the group consisting of a slurry, continuous flow bed, countercurrent extractor, moving bed, stationery bed, automated ion exchange system, an ion exchange column, and an impregnated filter.
